(19) United States
(12) Patent Application Publication
(10) Pub. No.: US 2022/0301697 A1
(43) Pub. Date: Sep. 22, 2022

(54) SYSTEM AND TECHNIQUES FOR INVENTORY DATA RECONCILIATION

(71) Applicant: VETSNAP CORP., Thousand Oaks, CA (US)

(72) Inventors: Steven P. ELLIOTT, Thousand Oaks, CA (US); Yangyang SHI, Thousand Oaks, CA (US); Gregory A. SCHIMPFF, Thousand Oaks, CA (US)

(21) Appl. No.: 17/540,803

(22) Filed: Dec. 2, 2021

Related U.S. Application Data
(60) Provisional application No. 63/162,918, filed on Mar. 18, 2021.

(51) Int. Cl. G16H 40/20; G06Q 30/00

(52) U.S. Cl. CPC G16H 40/20; G06Q 30/018

(57) ABSTRACT

Systems and techniques for tracking and reconciling controlled substance usage data are disclosed. First and second sets of entries providing controlled substance usage information of a medical practice are obtained. The second set is obtained from a practice information management system associated with the medical practice. One or more inconsistencies between the first and second sets of entries is identified. The inconsistencies are reconciled based on one or more rules. A third set of entries incorporating the reconciliation into the first set of entries is generated.

### CROSS-REFERENCE TO RELATED APPLICATIONS

This patent application claims priority to U.S. Provisional Patent Application Ser. No. 63/162,918, filed Mar. 18, 2021, entitled “A process to reconcile the controlled substance usage log that automatically compares the entries to client/patient drug usage data that can be housed in a veterinary clinic's practice management software,” which is herein incorporated by reference in its entirety.

### FIELD

Embodiments disclosed herein generally relate to practice management and inventory systems, and more specifically, to tracking and reconciling controlled substance usage data.

### BACKGROUND

In the veterinary industry, a practice (e.g., a clinic, hospital, pharmacy, etc.) must, by law, record and track usage of controlled substances in patients. Generally, controlled substances are chemicals, pharmaceutical agents, and the like that have been identified by a governmental body as having a potential for abuse. For example, the United States Drug Enforcement Agency categorizes and regulates controlled substances based on medicinal use, potential for abuse, and safety or dependence liability.

Tracking usage of controlled substances typically involves two sources: a controlled substance log and a practice information management system (PIMS). An individual at the practice (e.g., a veterinary doctor, assistant, or other staff) must input usage information in the controlled substance log as the usage occurs throughout the day. The PIMS maintains patient medical records, which includes itemized controlled drug substance usage for that patient entered generally for accounting purposes.

The controlled substance log is subject to audits to ensure that the veterinary practice is accurately tracking usage. Consequently, to maintain compliance with controlled substance laws, the practice must validate the data between the two sources to ensure no inconsistencies exist. However, current approaches for doing so rely on manual checks for missing entries, unmatched levels of usage, and other issues. Such approaches can be error prone and inefficient, as an individual generally reviews the controlled substance log and the PIMS side-by-side to identify and reconcile inconsistencies.

### SUMMARY

One embodiment presented herein discloses a computer-implemented method for managing controlled substance usage data. The method generally includes obtaining, by execution of one or more processors, a first set of entries. Each entry of the first set of entries provides controlled substance usage information at a medical practice. Each entry in the first set of entries includes a plurality of attributes and, for each attribute of the plurality of attributes, a corresponding value. The method also generally includes obtaining, from a practice information management system associated with the medical practice, a second set of entries, wherein each entry in the second set of entries provides controlled substance usage information at the medical practice, and wherein each entry includes the plurality of attributes and, for each attribute, a corresponding value. One or more inconsistencies between the first set of entries and the second set of entries are identified. The identified inconsistencies are reconciled based on one or more rules. A third set of entries is generated, which incorporates the reconciliation of the identified inconsistencies into the third set of entries.

Another embodiment presented herein discloses a system for managing controlled substance usage. The system includes a data aggregation server comprising one or more first processors and a first memory storing a plurality of first instructions. The system further includes a platform server comprising one or more second processors and a second memory storing a plurality of second instructions. The system yet further includes a client device comprising one or more third processors and a third memory storing a plurality of third instructions and a first set of entries. Each entry of the first set of entries provides controlled substance usage information at a medical practice, and each entry in the first set of entries includes a plurality of attributes and, for each attribute of the plurality of attributes, a corresponding value. The first plurality of instructions, when executed by the one or more first processors, causes the data aggregation server to obtain, from a practice information management system associated with the medical practice, data providing controlled substance usage information at the medical practice; convert the data into the second set of entries, wherein each entry includes the plurality of attributes and, for each attribute, a corresponding value, wherein the second set of entries is of the same format as the first set of entries; and transmit the second set of entries to the platform server. The second plurality of instructions, when executed by the one or more second processors, causes the platform server to retrieve the first set of entries from the client device; receive the second set of entries from the data aggregation server; identify one or more inconsistencies between the first set of entries and the second set of entries; reconcile, based on one or more rules, the identified one or more inconsistencies; and generate a third set of entries incorporating the reconciliation of the identified one or more inconsistencies into the first set of entries.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 illustrates a simplified conceptual diagram of an example embodiment computing environment in which controlled substance usage data from multiple sources is tracked and reconciled;

FIG. 2 is a simplified block diagram of an example embodiment of the client device described relative to FIG. 1;

FIG. 3 is a simplified block diagram of an example embodiment of the data aggregation server described relative to FIG. 1;

FIG. 4 is a simplified block diagram of an example embodiment of the digital log server described relative to FIG. 1;

FIG. 5 is a simplified conceptual diagram of an example inventory management application interface depicting a summary comparison of controlled substance usage logs from multiple sources;

FIG. 6 is a simplified conceptual diagram of an example inventory management application interface depicting a controlled substance usage log entry;

FIG. 7 is a simplified flow diagram of an example method for tracking and reconciling controlled substance usage data; and

FIG. 8 is a simplified flow diagram of an example method for reconciling inconsistencies between controlled substance usage data of multiple sources.

### DETAILED DESCRIPTION

Embodiments presented herein disclose systems and techniques for tracking and reconciling controlled substance usage data. More particularly, embodiments provide a multi-source validation and reconciliation approach that identifies, reports, and manages inconsistencies between controlled substance usage data of each source. To do so, the approach uses various attributes associated with usage data commonly provided in such independent sources, including drug name, date of usage, client, patient chart number, and so on, to cross-reference between sources. Based on the type of inconsistency in the usage data between sources, the approach can automatically correct the inconsistency (e.g., in minor inconsistencies such as misspellings, errors with species name, etc.) or flag for further review by a user (e.g., in inconsistencies pertaining to usage quantity, time of use, etc.).

Further, these embodiments may be adapted to an inventory management system platform that integrates with a practice inventory management system (PIMS) of a medical or veterinary practice. The platform may serve as one source of controlled usage data, providing a controlled substance usage log that the veterinary practice can use (e.g., via a client device executing a platform application thereon) to enter standardize usage data throughout the day while treating patients. The platform may also obtain, aggregate, and standardize data obtained from the PIMS to more efficiently validate data sets between the platform controlled substance usage log and the PIMS data. For instance, to efficiently validate usage data between the controlled substance log of the platform and the PIMS, the platform architecture and PIMS may be arranged such that discrete functions of the validation process (e.g., are carried out individually in distinct computer systems of the platform, such that computational resources of a single system can be allocated to a single function. Once validated, a resulting reconciled usage log can be generated and output to the platform application.

Advantageously, the techniques described herein validate multi-source usage data relatively efficiently and more accurately compared to preexisting manual approaches of comparing two electronic (or a combination of physical and electronic) lists side-by-side. In addition, standardizing PIMS data allows the platform to more accurately validate controlled substance data with the PIMS data, further allowing for efficient use of computational resources, and further still, allowing for integration of any number of PIMS with the platform.

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

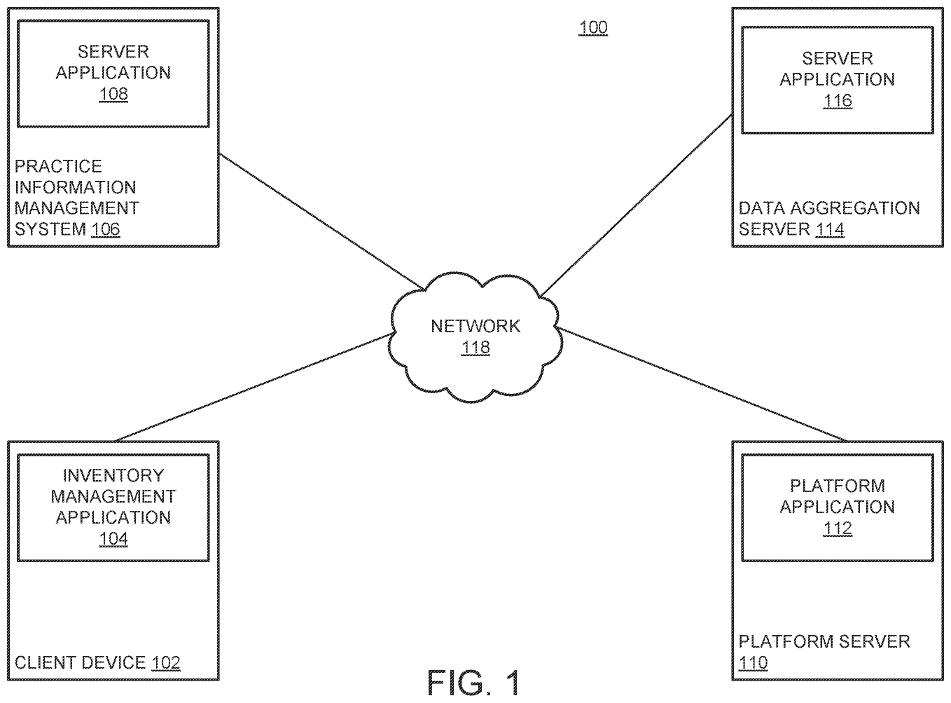

Referring now to FIG. 1, a computing environment 100 in which controlled substance usage data from multiple sources is tracked and reconciled, according to an embodiment. As shown, the computing environment 100 includes a client device 102, a practice information management system (PIMS) 106, a platform server 110, and a data aggregation server 114, each interconnected via a network 118 (e.g., the Internet, a local area network, private network, etc.).

The client device 102 may be embodied as a physical computing device (e.g., a desktop computer, laptop computer, mobile device such as a mobile tablet or a smartphone, etc.) or a virtual computing instance executing in a cloud network. A user of the client device 102 may be an individual employed at a medical practice, such as a veterinary practice (e.g., a veterinary clinic, hospital, office with a pharmacy, etc.) that administers treatment to an animal patient, such as dispensing and administering medication to the patient, including controlled substances. As used herein, a controlled substance pertains to a drug or a chemical that is subject to governmental regulations with regard to manufacture, possession, and use.

Illustratively, the client device 102 includes an inventory management application 104. The inventory management application 104 provides an drug inventory management interface enabling the user to record controlled substance usage over the course of the day. The interface may be embodied as a “digital log book” that maintains records of usage and controlled substance inventory of the practice. For example, the user may, via the inventory management application 104, record entries of controlled substance usage into the digital log book provided by the interface. The interface may allow the user to enter, for example, patient information (e.g., name, chart ID, owner name, treatment), drug name, quantity, physician information, approval information, and so on. Further, the inventory management application 104 is integrated with one or more PIMS, such as PIMS 106.

The PIMS 106 may be a physical computing system (e.g., a desktop computer, workstation computer, laptop computer, etc.) or a virtual computing instance executing in the cloud. The PIMS 106 can also run on the premises of the veterinary practice or a remote location. As shown, the PIMS 106 further includes a server application 108, providing known functionalities of a PIMS, including managing patient data (including maintaining records of controlled substance usage per patient), invoicing, inpatient care workflows, reporting workflows, and so on.

In an embodiment, the inventory management application 104 is incorporated into an inventory management platform that includes one or more of the platform server 110 and the data aggregation server 114. In some embodiments, the data aggregation server 114 is a third-party server integrated with one or more components of the platform that retrieves, via the server application 116, PIMS data from the server application 108 and standardizes the data into a format that the inventory management application 104 can more easily interpret and further process. The server application 116 may integrate with the server application 108 of the PIMS 106 (e.g., via a module corresponding to the server application 116 incorporated into the server application 108). Further, the server application 116 may provide an application programming interface (API) to the platform application 112, allowing the platform application 112 to communicate and access functionalities provided by the data aggregation server 114. In other embodiments, one or a combination of the functions performed by the inventory management application 104, platform application 112, and server application 116 can be performed by a single application.

In an embodiment, the platform server 110 stores and maintains standardized PIMS data for later processing by the inventory management application 104. To do so, the platform server 110 includes a platform application 112 executing thereon that performs such functions. The platform application 112 may provide an API to the inventory management application 104 to enable communication (e.g., transmission of PIMS data, local controlled usage log data, etc.) between the applications 104 and 112. In other embodiments, the platform application 112 also maintains and stores usage data provided by multiple client devices 102 each executing an instance of the inventory management application 104 (e.g., in the event that a client device 102 is a mobile device such as a tablet, and the inventory management application 104 is operated by multiple users). In other further embodiments, the platform server 110 performs reconciliation of data recorded in the digital log book of the inventory management application 104 and the data provided in the PIMS 106. The platform application 112 may use APIs provided by the server application 116 to communicate with the PIMS 106.

As stated, controlled substance usage data may be recorded and maintained by the various components of the computing environment 100. For example, the inventory management application 104 maintains a controlled substance usage log in a digital log book. Further, the PIMS data includes controlled substance usage information. To maintain compliance with governmental regulations on controlled substances, it is important to ensure, in a timely manner, that controlled substance usage data across the various sources, including the inventory management application and the server application 108 of the PIMS 106, matches with one another. As further described herein, the platform application 112 (or the inventory management application 104) may validate controlled substance usage data of multiple sources and automatically correct or otherwise flag inconsistencies. To ensure this accuracy in the controlled substance usage data, the platform application 112 verifies and matches usage data of drugs being tracked from each individual source. The platform application 112 uses identifiable information such as drug name, date of usage, client, or patient chart number, and other typically recorded data. By finding possible matches and mismatches in usage quantities recorded in each source, the current approach saves a material amount of time for the user as well as computational resources in reconciling usage data.

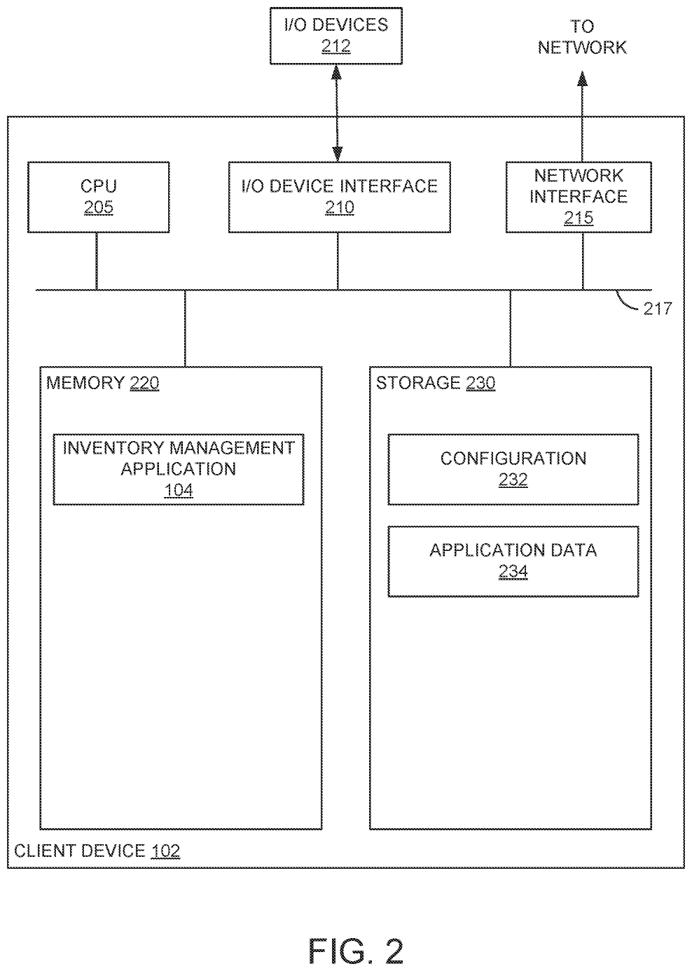

FIG. 2 further illustrates the client device 102. As shown, client device 102 includes, without limitation, a central processing unit (CPU) 205, an input/output (I/O) device interface 210, one or more I/O devices 212, a network interface 215, a memory 220, and a storage 230, each interconnected via a hardware bus 217. Of course, the actual client device 102 will include a variety of additional hardware components not shown. Additionally, in some embodiments, one or more of the illustrative components may be incorporated in, or otherwise form a portion of, another component.

The CPU 205 retrieves and executes programming instructions stored in the memory 220 (e.g., of the inventory management application 104). The CPU 205 may be embodied as one or more processors, each processor being a type capable of performing the functions described herein. For example, the CPU 205 may be embodied as a single or multi-core processor(s), a microcontroller, or other processor or processing/controlling circuit. In some embodiments, the CPU 205 may be embodied as, include, or be coupled to a field programmable gate array (FPGA), an application-specific integrated circuit (ASIC), reconfigurable hardware or hardware circuitry, or other specialized hardware to facilitate performance of the functions described herein. The hardware bus 217 is used to transmit instructions and data between the CPU 205, storage 230, network interface 215, and the memory 220. CPU 205 is included to be representative of a single CPU, multiple CPUs, a single CPU having multiple processing cores, and the like. The memory 220 may be embodied as any type of volatile (e.g., dynamic random access memory, etc.) or non-volatile memory (e.g., byte addressable memory) or data storage capable of performing the functions described herein. Volatile memory may be a storage medium that requires power to maintain the state of data stored by the medium. Non-limiting examples of volatile memory may include various types of random access memory (RAM), such as DRAM or static random access memory (SRAM). One particular type of DRAM that may be used in a memory module is synchronous dynamic random access memory (SDRAM). In particular embodiments, DRAM of a memory component may comply with a standard promulgated by JEDEC, such as JESD79F for DDR SDRAM, JESD79-2F for DDR2 SDRAM, JESD79-3F for DDR3 SDRAM, JESD79-4A for DDR4 SDRAM, JESD209 for Low Power DDR (LPDDR), JESD209-2 for LPDDR2, JESD209-3 for LPDDR3, and JESD209-4 for LPDDR4. Such standards (and similar standards) may be referred to as DDR-based standards and communication interfaces of the storage devices that implement such standards may be referred to as DDR-based interfaces.

The network interface 215 may be embodied as any hardware, software, or circuitry (e.g., a network interface card) used to connect the client device 102 over the network 118 and providing the network communication component functions described above. For example, the network interface 215 may be embodied as any communication circuit, device, or collection thereof, capable of enabling communications over the network 118 between the client device 102 and other devices (e.g., the PIMS 106, platform server 110, data aggregation server 114, etc.). The network interface 215 may be configured to use any one or more communication technology (e.g., wired, wireless, and/or cellular communications) and associated protocols (e.g., Ethernet, Bluetooth®, Wi-Fi®, WiMAX, 5G-based protocols, etc.) to effect such communication. For example, to do so, the network interface 215 may include a network interface controller (NIC, not shown), embodied as one or more add-in-boards, daughtercards, controller chips, chipsets, or other devices that may be used by the computing device 102 for network communications with remote devices. For example, the NIC may be embodied as an expansion card coupled to the I/O device interface 210 over an expansion bus such as PCI Express.

The I/O device interface 210 allows the I/O devices 212 to communicate with hardware and software components of the client device 102. For example, the I/O device interface 210 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some embodiments, the I/O device interface 210 may form a portion of a system-on-a-chip (SoC) and be incorporated, along with one or more of the CPU 205, the memory 220, and other components of the client device 102.

The I/O devices 212 may be embodied as any type of I/O device connected with or provided as a component to the client device 102. I/O devices such as keyboards, mice, and printers may be included as I/O devices 212 (e.g., to enter controlled substance usage information once administered to a patient). Illustratively, the memory 220 includes the inventory management application 104.

The storage 230 may be embodied as any type of devices configured for short-term or long-term storage of data such as, for example, memory devices and circuits, memory cards, hard disk drives (HDDs), solid-state drives (SSDs), or other data storage devices. The storage 230 may include a system partition that stores data and firmware code for the storage 230. The storage 230 may also include an operating system partition that stores data files and executables for an operating system.

As shown, the storage 230 includes a configuration 232 and application data 234. The configuration 232 may be embodied as any data indicative of user-definable parameters for managing the validation and reconciliation process. For example, in an embodiment, the configuration 232 may include one or more rules for handling inconsistencies once identified by the inventory management application 104. A given rule may identify which types of inconsistencies to automatically correct, e.g., minor inconsistencies in spellings of individual names. Another rule may identify types of inconsistencies to flag to a user for further review, e.g., inconsistencies in drug quantity administered to a patient. The application data 234 may be embodied as any type of data generated or maintained by the inventory management application 104, such as controlled substance usage data.

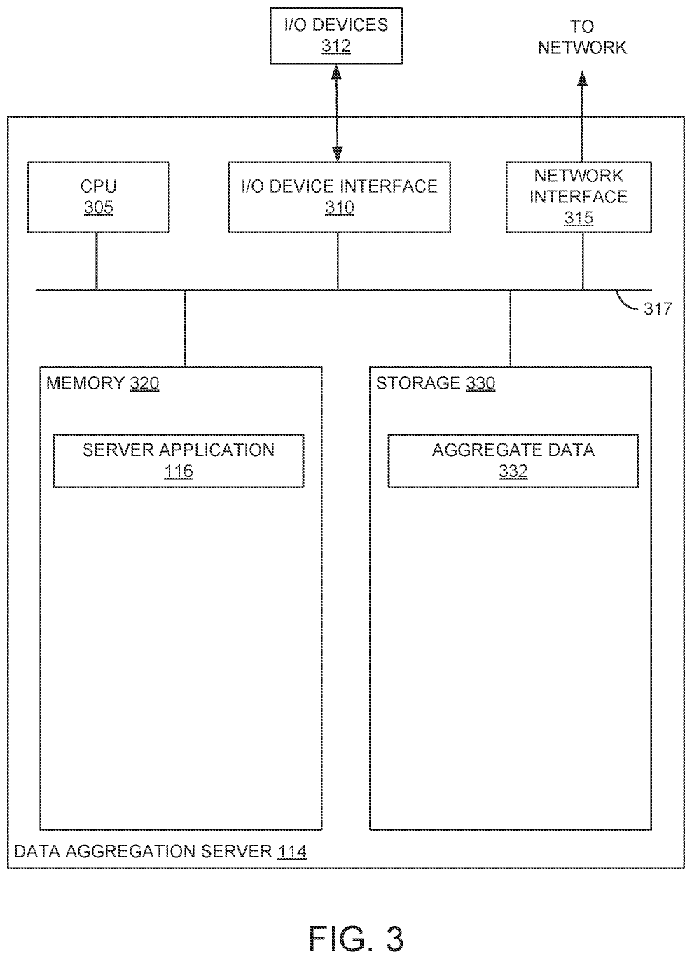

FIG. 3 further illustrates the data aggregation server 114. As shown, the data aggregation server 114 includes, without limitation, a CPU 305, an I/O device interface 310, one or more I/O devices 312, and a network interface 315, memory 320, and storage 330, each interconnected via a hardware bus 317. Of course, the actual data aggregation server 114 will include a variety of additional hardware components not shown. Additionally, in some embodiments, one or more of the illustrative components may be incorporated in, or otherwise form a portion of, another component. The CPU 305, I/O device interface 310, I/O devices 312, network interface 315, hardware bus 317, memory 320, and storage 330 are similar to the respective CPU 205, I/O device interface 210, I/O devices 212, network interface 215, hardware bus 217, memory 220, and storage 230 of the client device 102. Illustratively, the memory 320 includes the server application 116. The storage 330 includes aggregate data 332, which may be embodied as any type of data obtained from a variety of medical organizations (e.g., hospitals, veterinary centers, clinics, etc.) and aggregated by the server application 116.

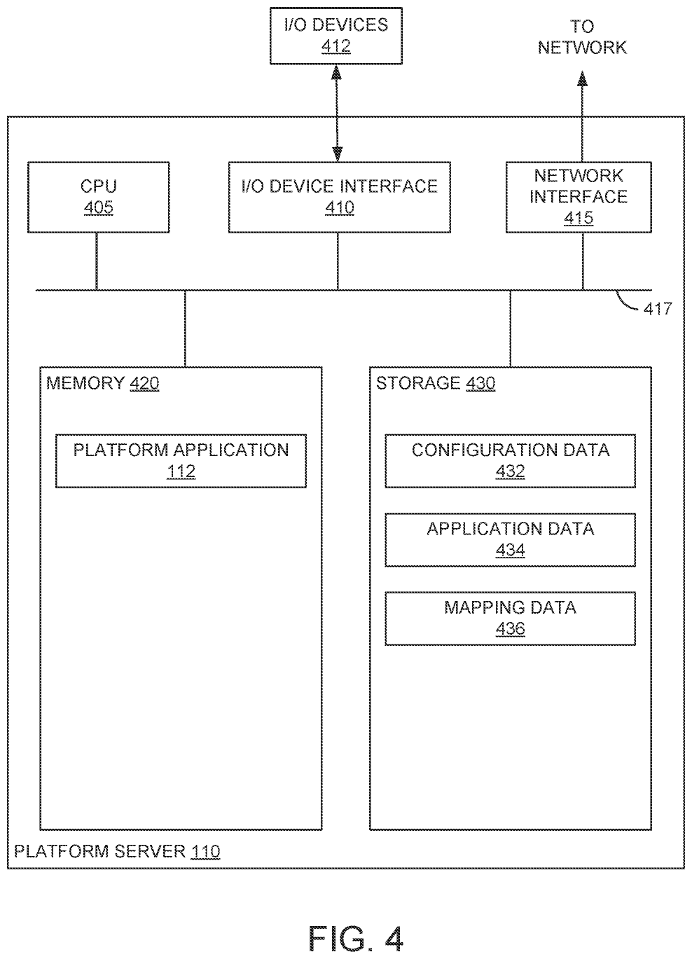

FIG. 4 further illustrates the platform server 110. As shown, the platform server 110 includes, without limitation, a CPU 405, an I/O device interface 410, one or more I/O devices 412, and a network interface 415, memory 420, and storage 430, each interconnected via a hardware bus 417. Of course, the actual platform server 110 will include a variety of additional hardware components not shown. Additionally, in some embodiments, one or more of the illustrative components may be incorporated in, or otherwise form a portion of, another component. The CPU 405, I/O device interface 410, I/O devices 412, network interface 415, hardware bus 417, memory 420, and storage 430 are similar to the respective CPU 205, I/O device interface 210, I/O devices 212, network interface 215, hardware bus 217, memory 220, and storage 230 of the client device 102. Illustratively, the memory 420 includes the platform application 112. The storage 430 includes mapping data 432. The mapping data 432 may be embodied as any type of data used to convert PIMS data obtained from the server application 108 to a format that the inventory management application 104, such as one or more schema. The storage 430 further includes configuration data 434, which may be embodied as any data indicative of user-definable parameters and rules for managing the validation and reconciliation process. The storage 430 also includes application data 436, which may be embodied as any type of data relating to standardized controlled substance usage data retrieved from the data aggregation server 114. In other embodiments, the application data 436 may include controlled substance usage logs sent by the inventory management application 104.

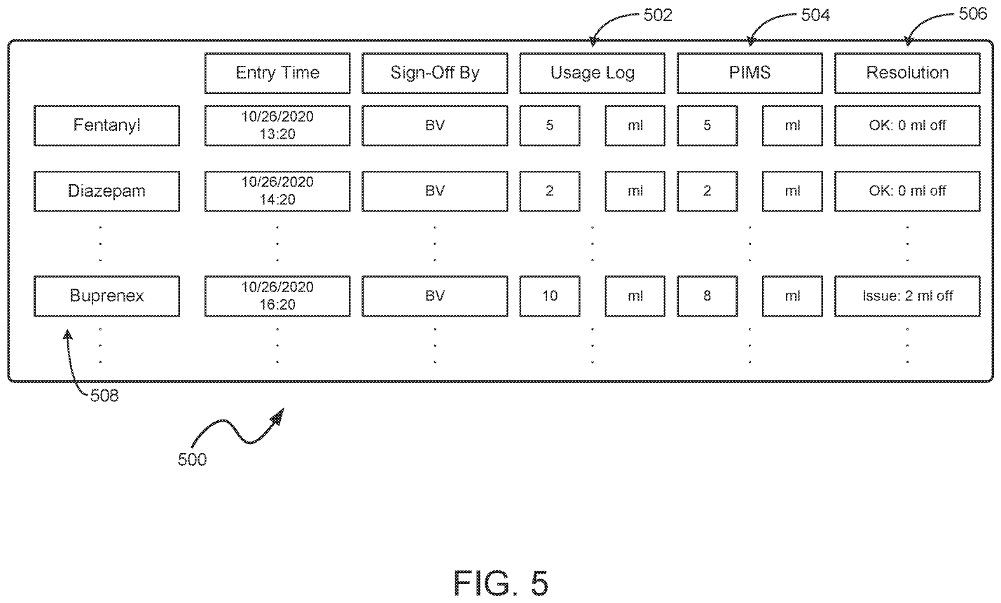

FIG. 5 illustrates an example reconciled controlled substance usage log 500 generated by the inventory management application 104. According to the techniques described herein, the platform application 112 (or the inventory management application 104) may generate the reconciled controlled substance usage log 500 as a result of an evaluation of two heterogeneous sources, such as data provided by the PIMS 106 and the log maintained by the inventory management application 104.

The example log 500 depicts a header row including usage attributes relating to a given controlled substance, the controlled substances being listed on the header row. Each row corresponds to one usage entry within the controlled substance log or an unmatched client or patient usage within the server application 108 to be reconciled due to an identified mismatch. As further shown, the attributes include information such as entry time (the time that the usage was entered into the given source) and initials of an individual signing off on the usage, and the corresponding rows include values corresponding to the attribute. In addition, the attributes also include drug quantity columns 502 and 504, in which column 502 corresponds to drug quantities obtained from a first source (a controlled substance usage log provided in the inventory management application 104 that is used to track usage throughout a period of time) and in which column 504 corresponds to drug quantities obtained from a second source (controlled substance usage data obtained via the PIMS 106).

The log 500 also provides a column 506 indicating a resolution between the preceding columns 502 and 504. As shown, the first two entries for Fentanyl and Diazepam provide a resolution of “OK: 0 ml off,” which indicates that the drug quantities matched with one another. By contrast, entry 508 for Buprenex shows an inconsistency between the drug quantity values entered in columns 502 and 504, in which “10 ml” is recorded for column 502 and “8 ml” is recorded for column 504. The column 506 flags this issue for resolution, indicating “Issue: 2 ml off.” In response, a user of the inventory management application 104 can use this information to determine the correct amount administered and resolve the issue within the application 104, which in turn propagates the resolution to the PIMS 106 and the platform server 110.

As further described herein, the log 500 may also provide additional information relating to inconsistencies other than drug quantities between the sources. For example, the log 500 may specify misspellings, differences in sign-off individuals, differences in entry time, and the like.

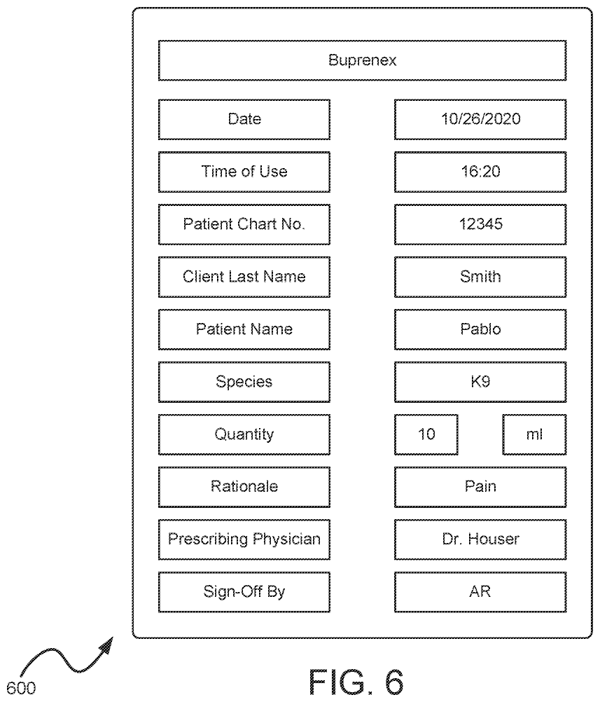

FIG. 6 illustrates an example list 600 of data included in a controlled substance usage log maintained in the inventory management application 104. The list 600 is an example of an entry that may be viewable in an interface of the inventory management application 104. Illustratively, the list 600 includes a variety of attributes, such as date, time of use, patient chart number, client last name, patient name, species, quantity, rationale, prescribing physician, and initials of the individual who signed off. The list 600 also includes corresponding values to those attributes to the right-hand side thereof. The platform application 112 may use these attributes as common identifier data to cross-reference the respective values between sources including the inventory management application 104 and the PIMS 106.

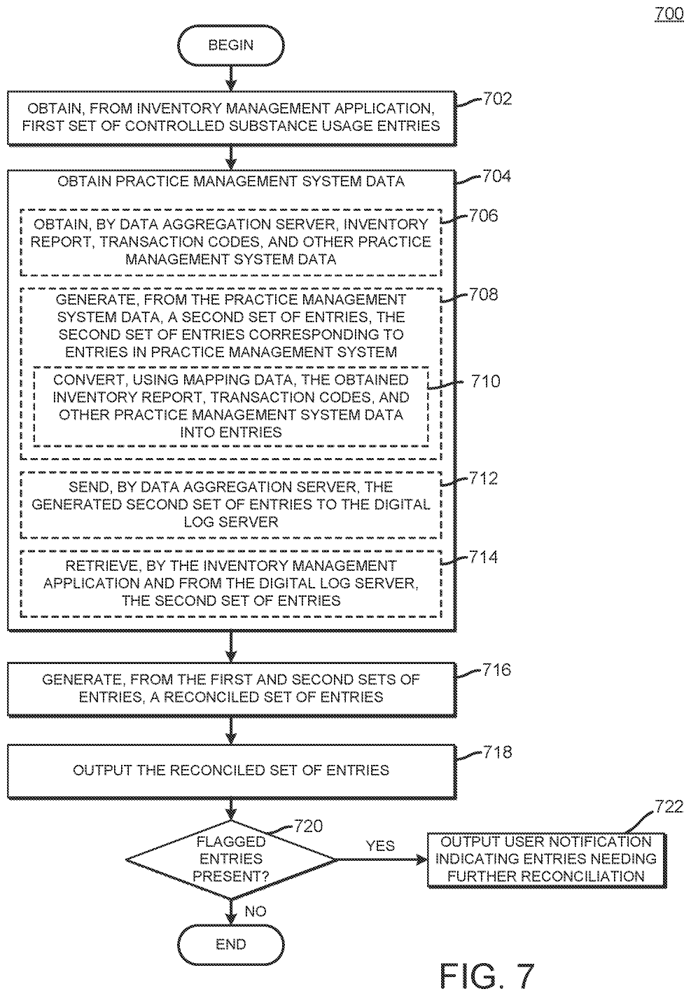

FIG. 7 illustrates an example method 700 for tracking and reconciling controlled substance usage data, according to an embodiment. In this example, the method 700 is carried out by the platform application 112. However, the method 700 can be performed by a variety of systems managing controlled substance usage data, including the inventory management application 104 and the server application 108 of the PIMS 106.

As shown, the method 700 begins in block 702, in which the platform application 112 obtains a first set of controlled substance usage entries from the inventory management application 104. For example, a user of the inventory management application 104 may initiate the a validation and reconciliation option via a user interface provided by the inventory management application 104, which in turn transmits the first set of controlled substance usage entries to the platform application 112. As another example, the platform application 112 may periodically perform the method 700 at predefined intervals (e.g., every hour, end of day, end of week, etc.) and retrieve the first set of controlled substance usage entries from the inventory management application 104 automatically. In this example method, the first set of controlled substance usage entries corresponds to the controlled substance usage log maintained by the inventory management application 104.

In block 704, the platform application 112 obtains practice management system data. For example, to do so, in block 706, the platform application 112 may cause the server application 116 of the data aggregation server 114 to send a request to the server application 108 of the PIMS 106 for such data, which can include an inventory report, transaction codes, transaction records, and other data that a PIMS typically manages. Once authorized, the server application 116 may retrieve the data from the PIMS 106. In block 708, the server application 116 generates, from the retrieved data, a second set of controlled substance usage entries. For example, to do so, in block 710, the server application 116 converts, from aggregate data 332, the PIMS data to a standardized controlled substance usage log that can be interpreted by the inventory management application 104. The platform application 112 may subsequently use mapping data 432 to evaluate the converted data (e.g., by a schema provided by the mapping data 432 linking correspondences between a given type in the PIMS data, such as a transaction code, to a type used in the controlled substance log of the inventory management application 104.

In block 712, the server application 116 may send the generated second set of entries to the platform server 110. In turn, the platform application 112 may store the second set of entries. In block 714, the inventory management application 104 retrieves the second set of entries from the platform server 110.

In other embodiments, blocks 704 through 714 may be carried out individually by the platform application 112 or the inventory management application 104. For example, the inventory management application 104 may obtain PIMS data directly from the server application 108 of the PIMS 106 and convert the PIMS data to the second set of controlled substance usage entries. Doing so may potentially incur an additional computational cost by the client device 102. Offloading standardization of the PIMS data to a separate system, such as the data aggregation server 114 and storage at the platform server 110 may preserve resources and achieve improved efficiency at the client device 102, such as in cases in which the client device 102 is resource-constrained. In addition, offloading the standardization to the data aggregation server 114 preserves computational resources at the platform server 110.

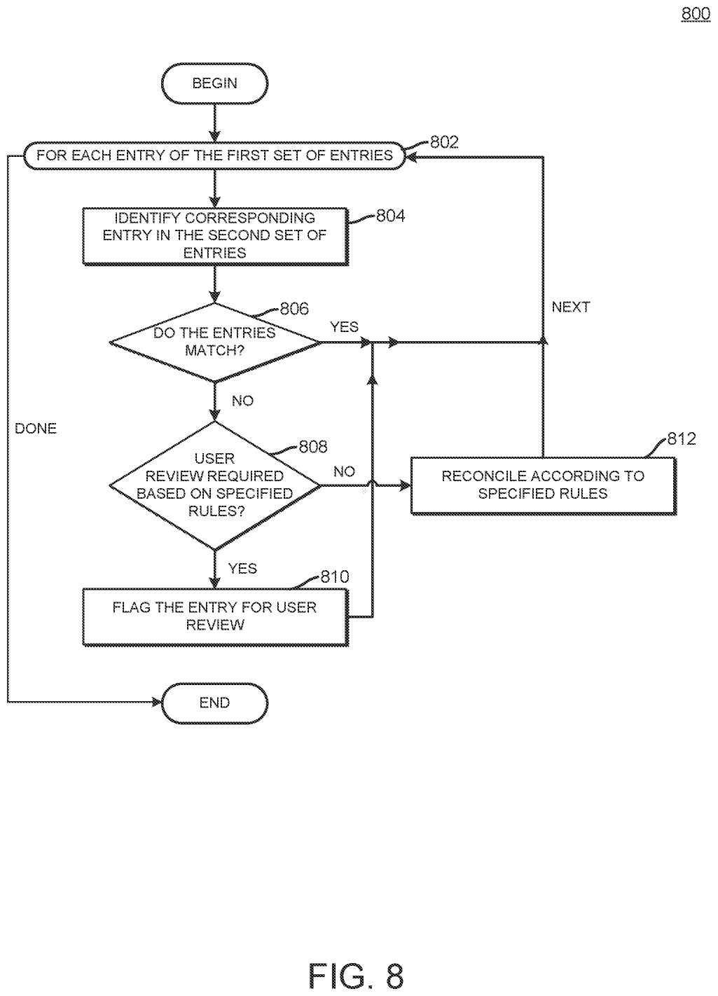

Continuing to block 716, the platform application 112 generates, from the first and second sets of entries, a reconciled set of entries. This process is described further herein with regard to FIG. 8. The reconciled set of entries corresponds to a distinct controlled substance usage log, in which inconsistencies between the first and second sets of entries have been identified and corrected and/or flagged for further review.

In block 718, the platform application 112 may output the reconciled set of controlled substance usage entries, e.g., to the client device 102, which in turn presents the output to a display of the client device 102 via the inventory management application 104. The output may present each entry and a status associated with that entry. For example, the status may indicate whether the a given entry matched in both the first and second sets of entries, whether the entry did not match and was automatically corrected, and whether the entry did not match and was flagged for further review. In the event of a mismatch, the output may also include the discrepancies between the respective entries in the first and second sets of entries. For example, the output may indicate that a drug quantity in the first set was entered at 10 mg and that the corresponding drug quantity in the second set was entered at 15 mg. In the event that the mismatch was automatically corrected, the output may also indicate the correction made. For example, the output may indicate that the mismatch pertained to an inconsistency of the patient species between the first and second sets. If automatically corrected, the output may specify which set the inventory management application 104 applied to the reconciled set.

A user can then review the reconciled data set and address discrepancies as needed and approve the finalized log. For example, the user can provide input to the inventory management application 104 to correct a given discrepancy. Once approved, the inventory management application 104 may propagate any corrections to either source of the first and second sets of entries. For example, the inventory management application 104 may transmit the generated reconciled set of entries or the finalized log to the platform server 110 and the PIMS 106.

FIG. 8 illustrates an example method 800 for reconciling inconsistencies between controlled substance usage data of multiple sources. As shown in block 802, the platform application 112 carries out the following steps for each entry in the first set of entries until complete. In block 804, the platform application 112 identifies a corresponding entry in the second set of entries. To do so, the platform application 112 may use uniquely identifying attributes provided in the first set of entries to query against the second set of entries. For example, the inventory management application 104 may use a patient ID, chart number, client ID, or some otherwise unique identifier.

In block 806, the platform application 112 determines whether the entries match one another. To determine a match, platform application 112 may compare the values associated with a given attribute between the sources. Further, the platform application 112 may use a variety of matching techniques in addition to identifying an exact match. For example, the platform application 112 may use a fuzzy algorithm, a best match algorithm, match scoring, and the like, e.g., definable in the configuration data 434. In such cases, the platform application 112 may apply a threshold in which, if a score indicating a likelihood that the two entries are a match exceeds the threshold, the platform application 112 classifies the entries as a match. In the event a match exists, the method 800 continues to the next entry in the first set of entries until complete.

Otherwise, if the entries do not match, then in block 808, the platform application 112 determines, according to one or more rules (e.g., specified in the configuration data 434), whether user review is required. For example, because drug quantities are heavily regulated, the rules may specify that differences in drug quantities between sources should be reviewed by an individual at the veterinary practice. In such a case, in block 810, the platform application 112 flags the entry for user review, and the method 800 proceeds to the next entry. As a contrasting example, other rules may specify that minor inconsistencies, such as spelling errors in a patient name may be automatically corrected according to one of the sources, as specified in the rules. In such a case, in block 812, the platform application 112 reconciles the entry according to specified rules.

Once each entry has been evaluated according to the method 800, the platform application 112 generates the reconciled set of controlled substance usage entries from the results.

### EXAMPLES

Illustrative examples of the technologies disclosed herein are provided below. An embodiment of the technologies may include any one or more, and any combination of, the examples described below.

Example 1 includes a computer-implemented method for managing controlled substance usage data, the method comprising obtaining, by execution of one or more processors, a first set of entries, wherein each entry of the first set of entries provides controlled substance usage information at a medical practice, and wherein each entry in the first set of entries includes a plurality of attributes and, for each attribute of the plurality of attributes, a corresponding value; obtaining, from a practice information management system associated with the medical practice, a second set of entries, wherein each entry in the second set of entries provides controlled substance usage information at the medical practice, and wherein each entry includes the plurality of attributes and, for each attribute, a corresponding value; identifying one or more inconsistencies between the first set of entries and the second set of entries; reconciling, based on one or more rules, the identified one or more inconsistencies; and generating a third set of entries incorporating the reconciliation of the identified one or more inconsistencies into the first set of entries.

Example 2 includes the subject matter of Example 1, and wherein obtaining, from the practice management system, the second set of entries comprises retrieving data from the practice information management system providing controlled substance usage information at the medical practice; and converting the data into the second set of entries, wherein the second set of entries is of the same format as the first set of entries.

Example 3 includes the subject matter of any of Examples 1 and 2, and wherein the data from the practice information management system comprises inventory report data and one or more transaction codes.

Example 4 includes the subject matter of any of Examples 1-3, and wherein identifying the one or more inconsistencies comprises determining whether each entry of the first set of entries matches a respective entry of the second set of entries.

Example 5 includes the subject matter of any of Examples 1-4, and wherein the respective entry of each entry of the first set of entries is determined based cross-referencing the value of one of the plurality of attributes of the first set of entries with the value of a corresponding one of the plurality of attributes of the second set of entries.

Example 6 includes the subject matter of any of Examples 1-5, and wherein determining whether each entry of the first set of entries matches the respective entry of the second set of entries comprises applying a fuzzy matching algorithm to the entry and the respective entry.

Example 7 includes the subject matter of any of Examples 1-6, and wherein determining whether each entry of the first set of entries matches the respective entry of the second set of entries comprises applying scoring threshold to the entry and the respective entry.

Example 8 includes the subject matter of any of Examples 1-7, and wherein identifying the one or more inconsistencies further comprises, upon determining that the entry does not match the respective entry, applying one or more rules to determine whether to automatically correct the entry.

Example 9 includes the subject matter of any of Examples 1-8, and further including, flagging the entry for user review.

Example 10 includes the subject matter of any of Examples 1-9, and further including, receiving input from the user to correct the entry.

Example 11 includes the subject matter of any of Examples 1-10, and further including, updating the practice information management system with the third set of entries.

Example 12 includes the subject matter of any of Examples 1-11, and further including, outputting the generated third set of entries to a display of a device.

Example 13 includes the subject matter of any of Examples 1-12, and further including, outputting the identified one or more consistencies to the display.

Example 14 includes the subject matter of any of Examples 1-13, and wherein the medical practice is a veterinary practice.

Example 15 includes a system for managing controlled substance usage, comprising a data aggregation server comprising one or more first processors and a first memory storing a plurality of first instructions; a platform server comprising one or more second processors and a second memory storing a plurality of second instructions; and a client device comprising one or more third processors and a third memory storing a plurality of third instructions and a first set of entries, wherein each entry of the first set of entries provides controlled substance usage information at a medical practice and wherein each entry in the first set of entries includes a plurality of attributes and, for each attribute of the plurality of attributes, a corresponding value, wherein the first plurality of instructions, when executed by the one or more first processors, causes the data aggregation server to obtain, from a practice information management system associated with the medical practice, data providing controlled substance usage information at the medical practice, convert the data into the second set of entries, wherein each entry includes the plurality of attributes and, for each attribute, a corresponding value, wherein the second set of entries is of the same format as the first set of entries, and transmit the second set of entries to the platform server, and wherein the second plurality of instructions, when executed by the one or more second processors, causes the platform server to retrieve the first set of entries from the client device, receive the second set of entries from the data aggregation server, identify one or more inconsistencies between the first set of entries and the second set of entries, reconcile, based on one or more rules, the identified one or more inconsistencies, and generate a third set of entries incorporating the reconciliation of the identified one or more inconsistencies into the first set of entries.

Example 16 includes the subject matter of Example 15, and wherein the data from the practice information management system comprises inventory report data and one or more transaction codes.

Example 17 includes the subject matter of any of Examples 15 and 16, and wherein to identify the one or more inconsistencies comprises to determine whether each entry of the first set of entries matches a respective entry of the second set of entries.

Example 18 includes the subject matter of any of Examples 15-17, and wherein the respective entry of each entry of the first set of entries is determined based cross-referencing the value of one of the plurality of attributes of the first set of entries with the value of a corresponding one of the plurality of attributes of the second set of entries.

Example 19 includes the subject matter of any of Examples 15-18, and wherein to determine whether each entry of the first set of entries matches the respective entry of the second set of entries comprises applying a fuzzy matching algorithm to the entry and the respective entry.

Example 20 includes the subject matter of any of Examples 15-19, and wherein to determine whether each entry of the first set of entries matches the respective entry of the second set of entries comprises applying scoring threshold to the entry and the respective entry.

1. A computer-implemented method for managing controlled substance usage data, the method comprising:
obtaining, by execution of one or more processors, a first set of entries, wherein each entry of the first set of entries provides controlled substance usage information at a medical practice, and wherein each entry in the first set of entries includes a plurality of attributes and, for each attribute of the plurality of attributes, a corresponding value;
obtaining, from a practice information management system associated with the medical practice, a second set of entries, wherein each entry in the second set of entries provides controlled substance usage information at the medical practice, and wherein each entry includes the plurality of attributes and, for each attribute, a corresponding value;
identifying one or more inconsistencies between the first set of entries and the second set of entries;
reconciling, based on one or more rules, the identified one or more inconsistencies; and
generating a third set of entries incorporating the reconciliation of the identified one or more inconsistencies into the first set of entries.

2. The computer-implemented method of claim 1, wherein obtaining, from the practice management system, the second set of entries comprises:
retrieving data from the practice information management system providing controlled substance usage information at the medical practice; and
converting the data into the second set of entries, wherein the second set of entries is of the same format as the first set of entries.

3. The computer-implemented method of claim 2, wherein the data from the practice information management system comprises inventory report data and one or more transaction codes.

4. The computer-implemented method of claim 1, wherein identifying the one or more inconsistencies comprises determining whether each entry of the first set of entries matches a respective entry of the second set of entries.

5. The computer-implemented method of claim 4, wherein the respective entry of each entry of the first set of entries is determined based cross-referencing the value of one of the plurality of attributes of the first set of entries with the value of a corresponding one of the plurality of attributes of the second set of entries.

6. The computer-implemented method of claim 4, wherein determining whether each entry of the first set of entries matches the respective entry of the second set of entries comprises applying a fuzzy matching algorithm to the entry and the respective entry.

7. The computer-implemented method of claim 4, wherein determining whether each entry of the first set of entries matches the respective entry of the second set of entries comprises applying scoring threshold to the entry and the respective entry.

8. The computer-implemented method of claim 4, wherein identifying the one or more inconsistencies further comprises, upon determining that the entry does not match the respective entry, applying one or more rules to determine whether to automatically correct the entry.

9. The computer-implemented method of claim 8, further comprising, flagging the entry for user review.

10. The computer-implemented method of claim 9, further comprising, receiving input from the user to correct the entry.

11. The computer-implemented method of claim 1, further comprising, updating the practice information management system with the third set of entries.

12. The computer-implemented method of claim 1, further comprising, outputting the generated third set of entries to a display of a device.

13. The computer-implemented method of claim 8, further comprising, outputting the identified one or more consistencies to the display.

14. The computer-implemented method of claim 1, wherein the medical practice is a veterinary practice.

15. A system for managing controlled substance usage, comprising:
a data aggregation server comprising one or more first processors and a first memory storing a plurality of first instructions;
a platform server comprising one or more second processors and a second memory storing a plurality of second instructions; and
a client device comprising one or more third processors and a third memory storing a plurality of third instructions and a first set of entries, wherein each entry of the first set of entries provides controlled substance usage information at a medical practice and wherein each entry in the first set of entries includes a plurality of attributes and, for each attribute of the plurality of attributes, a corresponding value,
wherein the first plurality of instructions, when executed by the one or more first processors, causes the data aggregation server to:
obtain, from a practice information management system associated with the medical practice, data providing controlled substance usage information at the medical practice,
convert the data into the second set of entries, wherein each entry includes the plurality of attributes and, for each attribute, a corresponding value, wherein the second set of entries is of the same format as the first set of entries, and
transmit the second set of entries to the platform server, and
wherein the second plurality of instructions, when executed by the one or more second processors, causes the platform server to:
retrieve the first set of entries from the client device,
receive the second set of entries from the data aggregation server,
identify one or more inconsistencies between the first set of entries and the second set of entries,
reconcile, based on one or more rules, the identified one or more inconsistencies, and
generate a third set of entries incorporating the reconciliation of the identified one or more inconsistencies into the first set of entries.

16. The system of claim 15, wherein the data from the practice information management system comprises inventory report data and one or more transaction codes.

17. The system of claim 15, wherein to identify the one or more inconsistencies comprises to determine whether each entry of the first set of entries matches a respective entry of the second set of entries.

18. The system of claim 17, wherein the respective entry of each entry of the first set of entries is determined based cross-referencing the value of one of the plurality of attributes of the first set of entries with the value of a corresponding one of the plurality of attributes of the second set of entries.

19. The system of claim 17, wherein to determine whether each entry of the first set of entries matches the respective entry of the second set of entries comprises applying a fuzzy matching algorithm to the entry and the respective entry.

20. The system of claim 17, wherein to determine whether each entry of the first set of entries matches the respective entry of the second set of entries comprises applying scoring threshold to the entry and the respective entry.
